(19) United States
(12) Patent Application Publication
(10) Pub. No.: US 2021/0309726 A1
(43) Pub. Date: Oct. 7, 2021

(54) TREATMENT OF MELANOMA BRAIN METASTASIS BY INHIBITION OF AMYLOID PRECURSOR PROTEIN CLEAVAGE

(71) Applicant: New York University, New York, NY (US)

(72) Inventors: Kevin KLEFFMAN, New York, NY (US); Eva HERNANDO-MONGE, New York, NY (US); Robert SCHNEIDER, New York, NY (US)

(21) Appl. No.: 17/051,413

(22) PCT Filed: May 21, 2019
(86) PCT No.: PCT/US19/33377

§ 371 Date: Oct. 28, 2020

Related U.S. Application Data
(60) Provisional application No. 62/674,417, filed on May 21, 2018.

(51) Int. Cl. C07K 16/18; A61P 35/04

(52) U.S. Cl. CPC C07K 16/18; A61P 35/04

(57) ABSTRACT

Provided are methods for treating cancer brain metastases by inhibiting amyloid precursor protein (APP) cleavage or the activity of the APP cleavage products, or both in the brain. The process may be inhibited by an inhibitor of α-, β-, or γ-secretase, or by an antibody specific for one or more of sAPPα, sAPPβ, CTF83, CTF99, p3, Aβ, and AICD.

### CROSS REFERENCE TO RELATED APPLICATIONS

This application claims priority to U.S. Provisional application No. 62/674,417, filed on May 21, 2018, the disclosure of which is incorporated herein by reference.

### BACKGROUND OF THE INVENTION

Melanoma, an aggressive cancer of neural crest origin, is the fifth most common cancer in the United States. Despite recent therapeutic advances, approximately two thirds of patients with Stage IV disease are eventually found to be unresponsive to therapy and succumb to their disease. The highest cause of morbidity and mortality in melanoma is melanoma brain metastasis. 40-50% of all patients with metastatic melanoma have clinically evident brain metastasis. Patients with brain metastasis are less responsive to therapy and have historically been excluded from clinical trials. Although some more recent trials allow patients with brain metastasis, highly restrictive enrollment criteria regarding the number, size and growth of brain metastasis leave most patients with brain metastasis without options.

Currently, melanoma brain metastasis is treated with immunotherapy, targeted therapy (BRAF inhibitors) and/or radiation therapy (usually whole brain radiation therapy). These therapies have variable success and when resistance develops, these patients have little to no options. There is currently no approved therapy that specifically targets brain metastasis in any cancer. Thus, brain metastasis remains an unmet therapeutic challenge.

### SUMMARY OF THE DISCLOSURE

The present disclosure is based on our findings that processing of amyloid precursor protein (APP) is required for metastatic melanoma cells to survive in the brain. These findings can be applied to any cancer brain metastases.

APP processing can generate a variety of products in the brain. Cleavage products generated by the actions of α-secretase and β-secretase are called secreted APP (sAPP) a or secreted APP (sAPP) (3, respectively. The carboxyterminal fragments (CTF) generated by α-secretase and β-secretase are called CTF83 and CTF99, respectively. γ-secretase cleavage of CTF83 and CTF99 results in the generation of p3 and amyloid β-peptide (Aβ), respectively, as well as the amino-terminal APP intracellular domain (AICD). Thus, the Aβ peptide, which is the predominant peptide implicated in Alzheimer's disease (AD), is generated by the sequential cleavages by β-secretase and γ-secretase enzymes. It is a 38 to 43 amino acid peptide.

This disclosure provides methods for inhibiting the survival and growth of metastatic cancer cells in the brain by inhibiting APP cleavage or the activity of the APP cleavage products, or both. The method comprises administering to an individual in need of treatment a therapeutically effective amount of one or more agents that inhibit APP processing, and/or the cleavage products of APP processing. The inhibitors may inhibit the activity of one or enzymes involved in APP processing or may inhibit the activity of one or more products of APP processing, or both. Examples of inhibitors of APP processing include antibodies directed to any of the APP enzymatic cleavage products and inhibitors of APP cleavage enzymes, including α-, β- and γ-secretases. The method can be used for inhibiting brain metastases associated with any cancer, including melanoma, lung cancer, breast cancer and the like.

In an embodiment, the method comprises inhibiting APP processing that generates the Aβ peptide. The Aβ peptide is found in the brain in two predominant forms—Aβ40 (amino acids 1-40) and Aβ42 (amino acids 1-42). The enzymes β- and γ-secretase are responsible for generation of Aβ40 and Aβ42 peptides. The use of the term Aβ or Aβ peptide in this disclosure includes both Aβ40 and Aβ42.

### BRIEF DESCRIPTION OF THE FIGURES

FIG. 1: Brain metastases (BM) vs Pair non-brain metastases (NBM) derived melanoma short term cultures (STCs) have increased ability to metastasize to the brain. 12-273 BM and NBM STCs were lentivirally labeled with GFP luciferase and introduced into NOG mice by intracardiac injection (A). Mice were monitored weekly by IVIS imaging and were sacrificed at day 37 due to weight loss. Representative IVIS images from Day 36 (B) are shown. 12-273 BM vs NBM had an increased brain-to-body luminescence ratio (C) and an increased number of brain metastatic foci (D) by histological analysis, demonstrating an increased brain-specific metastatic capability

FIG. 2: Proteomics analysis shows consistent alterations in proteins related to neurodegenerative pathologies in BM vs NBM STCs. A cohort of 14 brain metastasis (BM) and 11 non-brain metastasis (NBM) derived STCs, including 3 STC pairs, were analyzed by mass spectrometry-based proteomics. Key proteins related to neurodegeneration were found to be differentially expressed in BM vs pair NBM STCs (A, B). Select protein candidates from proteomics results were validated by western blot analysis in the STC pairs (C). Gene Set Enrichment Analysis of the proteomics from the whole cohort showed an enrichment in gene sets related to neurodegenerative pathologies (D, E).

FIG. 3: BM vs Pair NBM STCs have increased mitochondrial metabolism and APP processing. Analysis of STC pairs with Seahorse Mitostress test demonstrated increased basal oxidative phosphorylation (A) and decreased glycolysis (B) in BM vs pair NBM STCs (data from one pair shown). Quantification of mitochondrial length by electron microscopy was performed and showed increased mitochondrial fusion in BM vs pair NBM STCs (Representative Images C, Quantification D). Increased electron density of mitochondria in BM vs pair NBM STCs was also observed (not quantified). Substrate-specific in-vitro gamma-secretase assays were performed which demonstrated increased gamma-secretase cleavage of APP in BM vs NBM pair STCs (E, F). Interestingly, no consistent change in gamma-secretase cleavage of NOTCH was observed (G, H). Western blot analysis of whole cell lysate from STCs was performed with a C-Terminal APP Antibody which can recognize both full-length APP and the C-terminal fragments (CTFs) that remain in the membrane after APP is cleaved by alpha, beta, and/or gamma-secretase. Consistent with the results from the activity assay, APP-CTFs were consistently increased in BM vs pair NBM STCs while no consistent change was observed in cleaved NOTCH (I). No consistent change was observed in full-length APP (I). Secretion of Amyloid Beta (ABeta), which is produced by beta and gamma secretase cleavage of APP, was measured by ELISA. Secretion of both ABeta-40 (J) and Abeta-42 (K), the two predominant forms of ABeta, is increased in BM vs pair NBM STCs (data from one pair shown).

FIG. 4: APP is specifically required for melanoma to metastasize to the brain but not to other organs. APP was silenced by lentiviral shRNA in 12-273 BM STC and efficiency was verified by western blot analysis as compared to 12-273 BM lentiviral shRNA scramble (Scr) control (F). Silencing of APP had no significant effect on in-vitro proliferation (E). When introduced into NOG mice by intracardiac injection, silencing of APP dramatically inhibited the formation of brain metastasis without affecting metastasis to other organs (representative IVIS images B) as evidenced by a significantly decreased brain to body luminescence ratio (A). Skulls were analyzed by MRI post-mortem for detection of brain metastasis (representative images C,D). Organs were harvested and formalin-fixed, paraffin-embedded, and sectioned. Sections were stained by immunohistochemistry with an anti-NuMA antibody, which specifically recognizes human cancer cells within the mouse organs, and NuMA-positive cells were quantified using Visiopharm software. Results showed silencing of APP resulted in a severely reduced brain metastatic burden (representative images (H) and no significant difference in metastatic burden to the kidney or liver (G), demonstrating that APP has a specific, critical role in melanoma brain metastasis

FIG. 5: APP is required for growth and survival within the brain Parenchyma. 12-273 BM Scr and 12-273 BM shAPP were introduced into NOG mice by intracardiac injection. At various time points post-injection, mice were sacrificed and perfused with 4% PFA. Prior to sacrifice, mice underwent intracardiac injection with fluorescently labeled tomato-lectin, which specifically labels blood vasculature. Using a vibratome, mouse brains were sliced into 50 uM sections at resulting slices were stained for GFP, GFAP, and Cleaved Caspase-3 by free-floating immunofluorescence and slices were imaged by confocal microscopy. At day 1, melanoma cells are confined within the blood vasculature (B). From days 2-4, melanoma cells have a rounded morphology and extravasate across the blood brain barrier (BBB) into the brain parenchyma (C). About half of cells either do not extravasate across the BBB and die (G) or die shortly after extravasation (H), as evidenced the presence of cleaved caspase-3 staining. After extravasation, surviving cells spread out along the vasculature, a phenomenon known as vascular mimicry, and begin to divide (D). By day 14, cells have started to lose contact with the vasculature and divide in a rounded morphology, leading to the formation of micrometastases (E). By day 21, small macrometastes have formed as cells continue to divide and exponentially increase in number (F). As melanoma cells begin to form brain metastases, astrocytes become reactive and have increasing levels of direct contact with the melanoma cells leading to the formation of a “glial scar” circumscribing the metastasis (I-M, GFAP Staining Days 1-21). Quantification of melanoma cells over time demonstrated that silencing of APP inhibits melanoma cell growth and survival after day 7, but does not affect BBB invasion or early survival prior to day 7 (A). All images shown are from mice injected with 12-273 BM Scr.

FIG. 6: Silencing APP decreases the growth of pre-existing brain metastases. 12-273 BM Scr and 12-273 BM shAPP were cultured in absence of doxycycline and had similar levels of APP as measured by Western Blot (data not shown) and were introduced into NOG mice by intracardiac injection. At day 21, when small macrometastases have already formed as shown in FIG. 5, silencing of APP was induced by feeding the mice doxycycline-containing food. Silencing of APP in pre-formed brain metastases led to a significantly decreased brain metastatic burden at Day 36 (A—Representative IVIS images, B—Quantified Brain/Body luminescence), demonstrating that APP is a promising therapeutic target for the treatment of brain metastasis, including melanoma brain metastasis.

FIG. 7 shows the effect of anti Aβ antibody on progression of GFP luciferase melanoma cells in brain and body.

FIG. 8 shows APP is processed into amyloid beta by beta and gamma secretase cleavage. Probe-specific gamma secretase assays results for pair BM vs NBM STCs showing gamma secretase cleavage of APP (a) but not of NOTCH (b). Amyloid Beta-40 ELISAs were performed for the pair BM vs NBM STCs (c). SPA4CT-T43P—a heavily truncated form of APP which produces amyloid beta but not other APP-cleavage products—was designed (d). APP was knocked out using CRISPR-Cas9 and wild-type APP and SPA4CT-T43P were re-expressed and expression was verified by western blot (e). SPA4CT-T43P expression APP knockout cells showing amyloid beta secretion at comparable levels to NTC control cells (f). After intracardiac injection, APP knockout cells (sg-APP) show reduced ability to metastasize to the brain when compared with NTC control cells (g-h). Full length, wild-type APP re-expression in APP knockout cells was able to resuce the defects observed in brain metastasis (g-h). SPA4CT-T43P re-expression was also able to rescue the defects observed in brain metastasis with APP knockout (g-h).

### DESCRIPTION OF THE DISCLOSURE

This disclosure is based on the surprising finding that melanoma cells rely on APP processing for survival and growth in the brain. The disclosure provides compositions and methods useful for treatment of cancer brain metastasis, such as, for example, melanoma brain metastasis. The method comprises administering to an individual in need of treatment a composition comprising one or more inhibitors of APP processing and/or inhibitors of APP cleavage products. Our in-vivo findings that inhibiting APP cleavage or inhibiting its cleavage products can inhibit survival and growth of pre-existing melanoma brain metastases indicates that many of the existing therapeutics developed for AD (such as therapies that successfully cleared Aβ from the brain but did not show clinical benefit for AD), can be used for the treatment of patients with melanoma brain metastasis who have failed current available therapies and/or as a first-line combination treatment with existing therapies.

The term “APP processing” as used herein means enzymate cleavage of APP, such as, by one or more of α-secretase, β-secretase and γ-secretase.

An inhibitor of APP processing may inhibit one or more enzymes involved in APP cleavage. For example, an inhibitor may inhibit α-, β- and/or γ-secretases. Alternatively, or additionally, an inhibitor may inhibit the activity of one or more products of APP enzymatic cleavage. For example, an inhibitor may inhibit the activity of one or more of: sAPPα, sAPPβ, CTF83, CTF99, p3, Aβ, and/or AICD (referred to herein as APP cleavage products or APP-CP).

The amino acid sequences of APP and its cleavage products are well known in the art. If an accession number or UniProt database entry number is provided, the sequence from the database is incorporated herein by reference as it is available in the database as of the filing date of this application, and includes all versions of the sequence in the database, including any isoforms. The UniProt entry for APP is P05067 (incorporated herein by reference and includes sequences and isoforms in the database as of the filing date of the priority application).

Aβ40 sequence is
(SEQ ID NO: 1)
DAEFRHDSGYEVHHQKLVFFAEDVGSNKGAIIGLMVGGVV.
Aβ42 sequence is
(SEQ ID NO: 2)
DAEFRHDSGYEVHHQKLVFFAEDVGSNKGAIIGLMVGGVVIA.
sAPP-alpha sequence is:
(SEQ ID NO: 3)
LEVPTDGNAGLLAEPQIAMFCGRLNMHMNVQNGKWDSDPSGTKTCIDTK
EGILQYCQEVYPELQITNVVEANQPVTIQNWCKRGRKQCKTHPHFVIPY
RCLVGEFVSDALLVPDKCKFLHQERMDVCETHLHWHTVAKETCSEKSTN
LHDYGMLLPCGIDKFRGVEFVCCPLAEESDNVDSADAEEDDSDVWWGGA
DTDYADGSEDKVVEVAEEEEVAEVEEEEADDDEDDEDGDEVEEEAEEPY
EEATERTTSIATTTTTTTESVEEVVREVCSEQAETGPCRAMISRWYFDV
TEGKCAPFFYGGCGGNRNNFDTEEYCMAVCGSAMSQSLLKTTQEPLARD
PVKLPTTAASTPDAVDKYLETPGDENEHAHFQKAKERLEAKHRERMSQV
MREWEEAERQAKNLPKADKKAVIQHFQEKVESLEQEAANERQQLVETHM
ARVEAMLNDRRRLALENYITALQAVPPRPRHVFNMLKKYVRAEQKDRQH
TLKHFEHVRMVDPKKAAQIRSQVMTHLRVIYERMNQSLSLLYNVPAVAE
EIQDEVDELLQKEQNYSDDVLANMISEPRISYGNDALMPSLTETKTTVE
LLPVNGEFSLDDLQPWHSFGADSVPANTENEVEPVDARPAADRGLTTRP
GSGLTNIKTEEISEVKMDAEFRHDSGYEVHHQK
sAPP-beta sequence is:
(SEQ ID NO: 4)
LEVPTDGNAGLLAEPQIAMFCGRLNMHMNVQNGKWDSDPSGTKTCIDTK
EGILQYCQEVYPELQITNVVEANQPVTIQNWCKRGRKQCKTHPHFVIPY
RCLVGEFVSDALLVPDKCKFLHQERMDVCETHLHWHTVAKETCSEKSTN
LHDYGMLLPCGIDKFRGVEFVCCPLAEESDNVDSADAEEDDSDVWWGGA
DTDYADGSEDKVVEVAEEEEVAEVEEEEADDDEDDEDGDEVEEEAEEPY
EEATERTTSIATTTTTTTESVEEVVREVCSEQAETGPCRAMISRWYFDV
TEGKCAPFFYGGCGGNRNNFDTEEYCMAVCGSAMSQSLLKTTQEPLARD
PVKLPTTAASTPDAVDKYLETPGDENEHAHFQKAKERLEAKHRERMSQV
MREWEEAERQAKNLPKADKKAVIQHFQEKVESLEQEAANERQQLVETHM
ARVEAMLNDRRRLALENYITALQAVPPRPRHVFNMLKKYVRAEQKDRQH
TLKHFEHVRMVDPKKAAQIRSQVMTHLRVIYERMNQSLSLLYNVPAVAE
EIQDEVDELLQKEQNYSDDVLANMISEPRISYGNDALMPSLTETKTTVE
LLPVNGEFSLDDLQPWHSFGADSVPANTENEVEPVDARPAADRGLTTRP
GSGLTNIKTEEISEVKM
CTF99 sequence is:
(SEQ ID NO: 5)
DAEFRHDSGYEVHHQKLVFFAEDVGSNKGAIIGLMVGGVVIATVIVITL
VMLKKKQYTSIHHGVVEVDAAVTPEERHLSKMQQNGYENPTYKFFEQMQ
N
CTF83 sequence is:
(SEQ ID NO: 6)
LVFFAEDVGSNKGAIIGLMVGGVVIATVIVITLVMLKKKQYTSIHHGVV
EVDAAVTPEERHLSKMQQNGYENPTYKFFEQMQN
P3 sequence is:
(SEQ ID NO: 7)
LVFFAEDVGSNKGAIIGLMVGGVVIA
AICD sequence is:
(SEQ ID NO: 8)
IATVIVITLVMLKKKQYTSIHHGVVEVDAAVTPEERHLSKMQQNGYENP
TYKFFEQMQN

The sequences of any proteins or peptides provided in this disclosure include variants of the amino acid sequences that are at least 80%, 85%, 90%, 95%, 96%, 97%, 98% or 99% identical to the sequences of the proteins or peptides provided herein.

In one embodiment, the method comprises administering to an individual a composition comprising antibodies to one or more APP-CP. The antibodies can be human antibodies, humanized antibodies, or chimeric antibodies, which are reactive against one or more epitopes of APP cleavage products. The antibodies may be generated in response to administering an epitope of the APP-CP, or a peptide that is identical to a sequence within the APP-CP or a variant thereof (such as at least 85%, 90%, 95%, or 99% identical). The isolated antibodies or fragments thereof may be used without modifications, or they may be engineered, such as, for example, to generate chimeric or humanized antibodies or various fragments as described herein.

In an embodiment, the method comprises administering to an individual a composition comprising antibodies to Aβ or fragments thereof. The antibodies can be human antibodies, humanized antibodies, or chimeric antibodies, which are reactive against one or more epitopes of Aβ. The antibodies may be generated in response to administering an epitope of the Aβ peptide that is identical to a sequence within human Aβ or is a variant thereof (such as at least 85%, 90%, 95%, or 99% identical). The isolated antibodies or fragments thereof may be used without modifications, or they may be engineered, such as, for example, to generate chimeric or humanized antibodies or various fragments as described therein.

The term “antibody” (or its plural form) as used herein encompasses whole antibody molecules, full-length immunoglobulin molecules, such as naturally occurring full-length immunoglobulin molecules or full-length immunoglobulin molecules formed by immunoglobulin gene fragment recombinatorial processes, as well as antibody fragments. Antibody fragments can be fragments comprising at least one antibody-antigen binding site. The term “antibody” includes e.g. monoclonal, polyclonal, multispecific (for example bispecific), recombinant, human, chimeric, and humanized antibodies. The term “antibody” also encompasses minibodies, and diabodies, all of which preferably exhibit specific binding to an APP-CP, especially human APP-CP (such as Aβ) or an epitope fragment thereof. The term “antibody” also encompasses immunoglobulins produced in vivo, in vitro, such as, for example, by a hybridoma, and produced by synthetic/recombinant means. An antibody to an APP-CP may be modified by, for example, acetylation, formylation, amidation, phosphorylation, or polyethylene glycolation (PEGylation), as well as glycosylation.

Administration of APP-CP or antigenic fragments thereof can be used for generation of polyclonal antibodies. For example, suitable animals can be administered one or more APP-CP peptides or epitope containing fragments thereof and serum can be collected. Anti-APP-CP antibody-expressing cells can be isolated from immunized animals. The cells can be used for generation of hybridomas. Variable regions of antibody genes can be cloned from isolated cells by RT-PCR using the PIPE method (Dodev et al. (2014) Scientific Reports 4, 5885. doi:10.1038/srep058853). Recombinant human, humanized or chimeric mAbs can be constructed from these molecules and can be expressed and screened in functional and binding affinity assays.

The antibodies useful in the present methods may be whole immunoglobulin molecules such as polyclonal or monoclonal antibodies or may be antigen-binding fragments thereof, including but not limited to, Fab, F(ab′), F(ab′)2, Fv, dAb, Fd, CDR fragments, single-chain antibodies (scFv), bivalent single-chain antibodies, single-chain phage antibodies, diabodies, nanobodies and the like. The fragments of the antibodies may be produced synthetically or by enzymatic or chemical cleavage of intact immunoglobulins or may be genetically engineered by recombinant DNA techniques. These techniques are well known in the art.

The antibodies useful for the present method may be obtained from a human or a non-human animal. In many mammals, intact immunoglobulins have two heavy chains and two light chains. Each of the light chains is covalently linked to a heavy chain by a disulfide bond. The two heavy chains are linked to each other by additional disulfide bonds. The light chain typically has one variable domain (VL) and one constant domain (CL). The heavy chain can also have one variable domain (VH). The variable domains contain complementarity-determining regions (CDRs). The heavy chain can further have three or four constant domains (CHI, CH2, CH3 and CH4). The variability of the constant domains results is various isotypes such as IgA, IgD, IgE, IgG, and IgM.

The CDRs are primarily responsible for binding to an epitope of an antigen. The CDRs of each chain are typically referred to as CDR1, CDR2, and CDR3, numbered sequentially starting from the N-terminus, and are typically identified by the chain in which the particular CDR is located. Thus, a VH CDR3 (or VH-CDR3) is located in the variable domain of the heavy chain of the antibody in which it is found, whereas a VL CDR1 (or VL-CDR1) is the CDR1 from the variable domain of the light chain of the antibody in which it is found. An antibody that binds an APP cleavage product (e.g., Aβ), for example, will have a specific VH region and the VL region sequence, and thus specific CDR sequences. Antibodies with different specificities (i.e. different combining sites for different antigens) have different CDRs.

The terms VH or VH as used herein refer to the variable region of an immunoglobulin heavy chain, including a heavy chain of an Fv, scFv, dsFv or Fab, and the terms VL or VL refer to the variable region of an immunoglobulin light chain, including a light chain of an Fv, scFv, dsFv or Fab.

Single domain antibodies or nanobodies produced by camelids in response to introducing APP cleavage products (or peptide fragments thereof) into the camelids can also be used. The nanobodies are typically heavy chain antibodies and thus contain heavy chain homodimers and do not contain antibody light chains. These antibodies typically comprise a single variable domain and two constant domains (CH2 and CH3).

The term “monoclonal antibody” refers to an antibody produced by a single clone of B-lymphocytes or by a cell into which the light and/or heavy chain genes of a single antibody have been transfected. Monoclonal antibodies are produced by methods known to those of skill in the art, for instance by making hybrid antibody-forming cells from a fusion of myeloma cells with immune spleen cells. For example, mice (or other suitable animals) may be immunized with one or more isoforms of APP cleavage products (such as human Aβ) or a fragment thereof, and then ascites fluid samples can be collected. The samples can be screened and selected to develop a panel of monoclonal antibodies and corresponding hybridoma cell lines. Murine (or other) monoclonal antibodies can be isolated or generated and then humanized.

An antibody useful for the present method can be of any class. For example, an antibody of the present invention can be an antibody isotype IgG1, IgG2, IgG3, IgG4, IgM, IgA, IgD or IgE. For example, the antibody can be IgG2b. The term “isotype” as used herein, can in particular refer to the antibody class (such as e.g. IgG) that is encoded by heavy chain constant region genes. Sequences of human immunoglobulin constant regions are known in the art and are available in public databases such as National Center for Biotechnology Information (NCBI), U.S. National Library of Medicine.

The term “chimeric antibody” refers to an antibody which has framework residues from one species, such as human, and CDRs (which generally confer antigen binding) from another species, such as a murine antibody that specifically binds an APP cleavage product. In a chimeric antibody, some portions of the heavy and/or light chains may be identical or homologous to sequences from a particular species while other portions may be identical or homologous to sequences from a different species. Chimeric antibodies can be designed that generally exhibit decreased immunogenicity and increased stability. Techniques for cloning murine immunoglobulin variable domains known in the art—such as, for example, see Orlandi et al., Proc. Natl Acad. Sci. USA 86: 3833 (1989), and Leung et al., Hybridoma 13:469 (1994). As an example of a chimeric antibody, polynucleotides encoding the variable domains of the light chain or the heavy chain of an antibody derived from an animal (e.g., mouse, rat, or chicken) other than human can be linked to polynucleotides encoding the constant domains of the light chain or the heavy chain derived from a human antibody to produce a polynucleotide (such as DNA) encoding a chimeric antibody.

A “human” antibody (also called a “fully human” antibody) is an antibody that includes human framework regions and all of the CDRs from a single or different human immunoglobulins. Thus, frameworks from one human antibody can be engineered to include CDRs from a different human antibody. Methods for producing human antibodies are known in the art—such as, for example, see Mancini et al., 2004, New Microbiol. 27:315-28; Conrad and Scheller, 2005, Comb. Chem. High Throughput Screen. 8:117-26.

A “humanized antibody” is typically a human antibody that has one or more amino acid residues imported into it (i.e., introduced into it) from a source that is non-human. For example, a humanized antibody is a recombinant protein in which the CDRs of an antibody from a species such as rodent, rabbit, dog, goat, or horse are imported into human heavy and light variable domains. The constant domains (also referred to as framework regions) of the antibody molecule are generally the same as those of a human antibody. The non-human immunoglobulin providing the CDRs can be termed as “donor” and the human immunoglobulin providing the framework can be termed as “acceptor”. For example, all the CDRs can be from the donor immunoglobulin in a humanized immunoglobulin. Constant regions need not be always present, but if they are, they can be substantially identical to human immunoglobulin constant regions, i.e., at least about 85-90%, such as about 95% or more identical. A humanized antibody binds to the same antigen as the donor antibody that provides the CDRs. Humanized immunoglobulins can be constructed by means of genetic engineering (see for example, U.S. Pat. No. 5,585,089, and U.S. Publication No. 2010/0196266).

Antibody fragments can be produced by enzymatic digestion. For example, papain digestion of antibodies produces two identical antigen-binding fragments, called “Fab” fragments, and “Fc” fragment. The Fab fragment contains an entire L chain and the variable region domain of the H chain (VH), and the first constant domain of one heavy chain. Each Fab fragment is monovalent with respect to antigen binding, i.e., it has a single antigen-binding site. Pepsin treatment of an antibody yields a single large F(ab′)₂ fragment that roughly corresponds to two disulfide linked Fab fragments having divalent antigen-binding activity and is capable of cross-linking antigen. “Fv” is the minimum antibody fragment that contains a complete antigen-recognition and -binding site and single-chain Fv also abbreviated as “sFv” or “scFv” are antibody fragments that comprise the VH and VL antibody domains connected into a single polypeptide chain. The term “diabodies” refers to small antibody fragments prepared by constructing sFv fragments with short linkers between the VH and VL domains such that inter-chain but not intra-chain pairing of the V domains is achieved, resulting in a bivalent fragment, i.e., fragment having two antigen-binding sites. A single domain antibody (sdAb) is an antibody fragment which has a single monomeric variable antibody domain. ScAbs can be made from heavy-chain antibodies found in camelids. An antibody fragment can be a single variable region or a peptide consisting of or comprising a single CDR. A single-chain antibody has a heavy chain variable domain and a light chain variable domain linearly linked to each other via a linker.

The antibodies useful for treatment of melanoma brain metastasis can be bispecific or multispecific. Bispecific antibodies (diabodies) are antibodies that have binding specificities for at least two different epitopes of an antigen, such as two different epitopes of an APP-CP. For example, a polynucleotide (such as DNA) encoding a bispecific antibody can be produced by, for example, linking in order a polynucleotide encoding a heavy chain variable region A, a polynucleotide encoding a light chain variable region B, a polynucleotide encoding a heavy chain variable domain B, and a polynucleotide encoding a light chain variable domain A. Preferably, the heavy chain variable domain and the light chain variable domain are both derived from a human antibody.

An APP-CP specific antibody, or antigen-binding fragment thereof, can be administered to an individual in need of treatment at a dose that is effective to treat brain metastases. In general, suitable dosages for anti-APP cleavage product antibodies can range from about 0.1 mg/kg to 100 mg/kg. As an example, anti-Aβ antibodies for AD have been used at about 15-25 mg/kg depending on patient weight. Examples of dosages include 1, 5, 10, 15, 20, 25, 30, 35, 40, 45, 50, 55, 60, 65, 70, 75, 80, 85, 90, 95 and 100 mg/kg. A variety of dosage regimens are contemplated including dosage regimens in which the antibody may be administered repeatedly, e.g., on a daily, weekly or monthly schedule, over a short period or an extended period of time, e.g., months to years.

The present disclosure further provides pharmaceutical formulations suitable for use in the methods of treatment disclosed herein. The pharmaceutical formulations can be formulated for any convenient route of administration, e.g., parenteral or intravenous injection, and will typically include, in addition to the anti-APP cleavage product antibody, one or more acceptable carriers, excipients, and/or diluents suited to the desired mode of administration. In some embodiments, an antibody of the invention may be formulated for intravenous administration. In some embodiments, an antibody of the invention may be formulated in a suitable buffer, such as a phosphate buffer or an arginine buffer (such as an arginine succinate buffer). The buffer can contain one or more surfactants, e.g., a polysorbate.

In an embodiment, the antibody is directed to Aβ. Several anti-Aβ antibodies are known in the art and some have undergone clinical trials for treatment of Alzheimer's disease. Examples include Solanezumab (Eli Lilly, U.S. Pat. No. 7,195,761, incorporated herein by reference), Bapineuzumab (Pfizer, U.S. Pat. No. 6,750,324, incorporated herein by reference), aducanumab (Biogen), gantenerumab (Roche), and crenezumab (Genentech, See U.S. Patent Application No. 20170198030, incorporated herein by reference). In one embodiment, one or more of these antibodies may be used in the present methods to treat melanoma brain metastasis. Several other antibodies or modified antibodies are also described in the literature—See, for example, U.S. Patent Publication No. 20180051072 and 20180051071. One or more of the available antibodies may be used or further antibodies prepared as described herein. Examples of antibodies that bind to monomers of Aβ include bapineuzumab, solanezumab, gantenerumab, crenezumab and ponezumab. Examples of antibodies that only bind to oligomers or fibrils of Aβ include BAN2401, and aducanumab. Examples of BACE inhibitors include verubecestat, atabecestat, lanabecestat, elebecestat, CNP520. One skilled in the art will appreciate that humanized antibodies recognizing the same recognition sequence as a murine antibody that binds to amyloid beta (such as, for example, 4G8 or m266), or comprising the same CDRs as a murine antibody (or other animal antibodies) will bind to human amyloid beta. Similarly, humanized antibodies derived from other murine (or other animal) antibodies that bind to murine (or other animal) amyloid beta and recognizing the same recognition sequence(s), or comprising the same CDRs may also be used.

While the antibody may bind to any cleaved segment of APP, or any dimeric or oligomeric form of the cleavage products, in an embodiment, the antibody preferentially binds to the monomeric form of Aβ. An example of such an antibody is Solanezumab. In one embodiment, the antibody may bind to both the monomeric and the oligomeric form of Aβ. An example of such an antibody is Bapineuzumab.

In one embodiment, this disclosure provides a method for inhibiting the growth and/or survival of brain metastases by inhibiting the activity of α- β- and/or γ-secretase.

Examples of α-secretase inhibitors include, e.g., APH-0703, and ADAM (a disintegrin and metalloproteases) 10 inhibitors, such as G1254023X (GSK), and inhibitors of ADAM17. Examples of β-secretase inhibitors include BAN 2203, BAN2401 (Biogen/Eisai), CTS-21166 (CoMentis, Inc), E2609 (Elenbecestat), HPP-854 (Trans Tech Pharma), LY2811376 and LY2886721 (Eli Lilly).

Examples of γ-secretase inhibitors include the compounds disclosed in U.S. Patent publication no. 2014/0227173 to Eberhart et al., U.S. Patent publication no. 20110257163 to Palani et al., U.S. Patent publication no. 20110082153 to Aslanian et al., all incorporated herein by reference. Other known γ-secretase inhibitors are BMS-708163 (avagacestat) and ELND0005 (Elan). Because γ-secretase is known to have targets in addition to APP, one of which is the NOTCH family of transmembrane receptors, it is preferable to use an inhibitor of γ-secretase that preferentially or specifically inhibits the formation or accumulation of cleavage products of APP over any effects on NOTCH. One class of compounds that has been found to reduce Aβ without affecting NOTCH signaling includes the tyrosine kinase inhibitor imatinib mesylate (STI-571, trade name GLEEVEC) and compounds related thereto (Netzer et al., PNAS USA, 100:12444-12449, 2003, U.S. Patent publication no. 2004/0028673; WO2004/033925, all incorporated herein by reference).

In an embodiment, an individual in need of treatment may be administered one or more anti-APP cleavage product antibodies and one or more inhibitors of α-, β-, and/or γ-secretase. In an embodiment, an individual in need of treatment may be administered one or more anti-Aβ antibodies and one or more inhibitors of γ-secretase. The antibodies and the enzyme inhibitors may be administered as a single composition or as separate compositions, at the same time or at different times, over the same period of time or over different periods of time, and via the same route of administration or different routes of administration.

An individual in need of treatment for the present method is typically an individual who has been diagnosed with cancer, such as for example, melanoma. The individual may be diagnosed as an individual having melanoma wherein the melanoma has metastasized to brain. Diagnosis of metastasis of melanoma to brain is generally carried out by one or more of various types of brain scans including MRI, CT, and PET. Patients with neurologic signs and symptoms generally undergo scans for disease diagnosis. Stage IV/high risk stage III patients without neurological symptoms may also undergo scans.

The present method may be used in a patient in whom brain metastasis has been diagnosed or may be used on an individual who is considered to be at high risk of developing brain metastasis. For example, advanced stage melanoma patients or advanced stage lung cancer or breast cancer patients may be identified as being suitable candidates for the present prophylactic therapy.

The individual in need of treatment may be an individual who is being treated for melanoma (or other cancers) by radiation therapy, such as whole brain radiation therapy (WBRT), BRAF (B-raf) inhibitors (such as Vemurafenth (Zelboraf) and dabrafenib (Tafinlar)), and/or immunotherapy, or by a chemotherapeutic drug, such as, for example, doxorubicin, cyclophosphamide, dacarbazine (DTIC), hydroxylurea, temozolomide, cisplatin, carboplatin, camptothecins, etoposide, vinblastine, Actinomycin D and cloposide. As such, the method of the present disclosure to inhibit the growth and survival of metastatic cells in the brain may be used in conjunction with treatment of the underlying melanoma or other cancers. For example, in an embodiment, the present method may comprise subjecting an individual to WBRT or immunotherapy, or administering to the individual a composition comprising a BRAF inhibitor or chemotherapeutic agent that is effective for reducing or inhibiting the growth of melanoma, and administering to the individual a composition comprising an agent that inhibits the growth and/survival of brain metastatic cells. The two approaches (treatment of underlying melanoma (or other cancers) and inhibition of growth/survival of brain metastasis) may be carried out concurrently, or sequentially. In an embodiment, the treatment directed at inhibition of melanoma (or other cancers) may be started first and then the composition comprising the inhibitor of APP processing or anti-APP-CP antibody may be started subsequently. The administration regimens for the two treatments may overlap and may be continued over a period of days, weeks or months, as needed.

The present methods may be used for brain metastasis associated with any cancer. Common examples include melanoma, lung cancer, and breast cancer.

The following examples are provided as illustrative examples and are not intended to be restrictive in any way.

### Example 1

This example demonstrates that APP processing is required for survival of metastatic melanoma cells in the brain. Through a high throughput proteomics screen of brain metastasis (BM) and non-brain metastasis (NBM) derived melanoma short term cultures (STCs), we identified consistent alterations in proteins related to neurodegenerative pathologies in BM vs NBM STCs. In STC pairs (a BM and a NBM STC both derived from the same patient), we show that BM STCs have increased APP-specific gamma secretase activity while lacking consistently increased gamma secretase cleavage of NOTCH. We silenced APP in a melanoma STC and analyzed its metastatic ability in-vivo by intracardiac injection into immunocompromised mice. When compared to a control, silencing of APP dramatically reduced the amount of brain metastasis without having any significant effect on metastasis to other organs, such as the liver. By sacrificing mice at various early time points post-intracardiac injection, we have determined that silencing of APP does not affect early events in brain metastasis, such as homing to the brain vasculature and blood brain barrier invasion, but rather affects survival and growth at later times. Using an inducible shRNA, we induced silencing of APP 21 days post intracardiac injection—a time at which we show that brain metastases have already formed in our model. These results show that silencing of APP in preformed brain metastases resulted in significantly reduced brain metastatic burden as compared to a control. Further, mutant rescue experiments demonstrated that reintroduction of amyloid beta is sufficient to rescue loss of brain metastasis seen with APP knockout.

In contrast to Botelho et al., (J Invest Dermatol. 2010 May; 130(5):1400-10. doi: 10.1038/jid.2009.296. Epub 2009 Sep. 17), we did not observe the mild decrease in proliferation in-vitro when we silenced APP, nor did we observe changes in morphology or pigmentation. Additionally, unlike the findings of Strilic et al (Nature, 2016 Aug. 11; 536(7615):215-8. Epub 2016 Aug. 3), which describe a role for APP in lung metastasis by inducing necroptosis in endothelial cells to facilitate extravasation, we show that silencing of APP only affects survival and growth of melanoma cells within the brain parenchyma and has no effect on extravasation across the blood brain barrier.

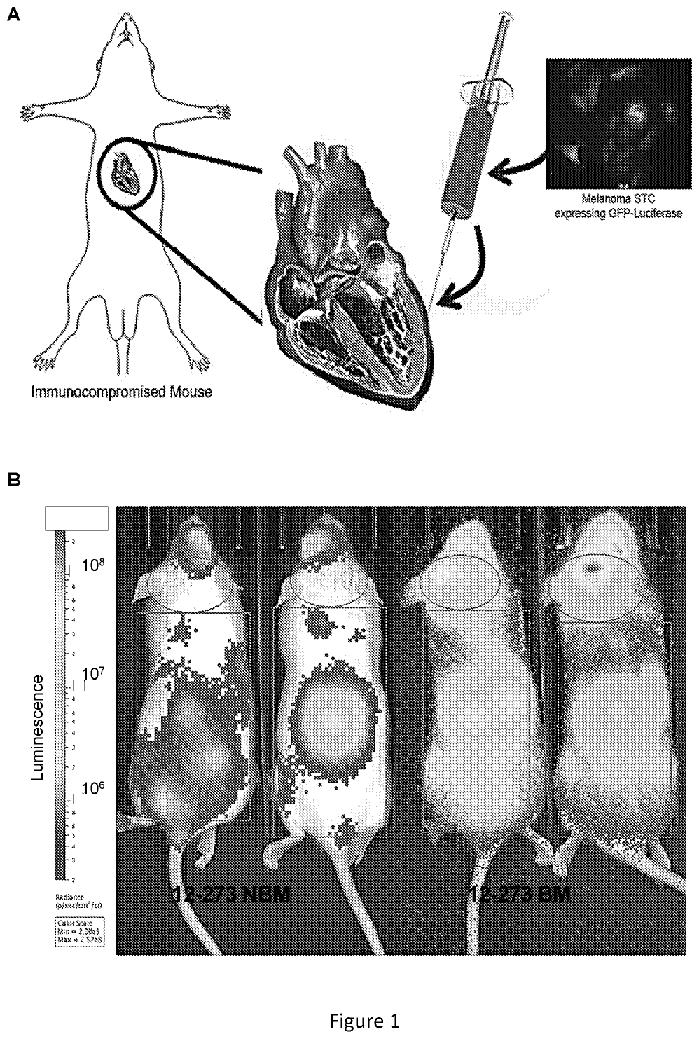

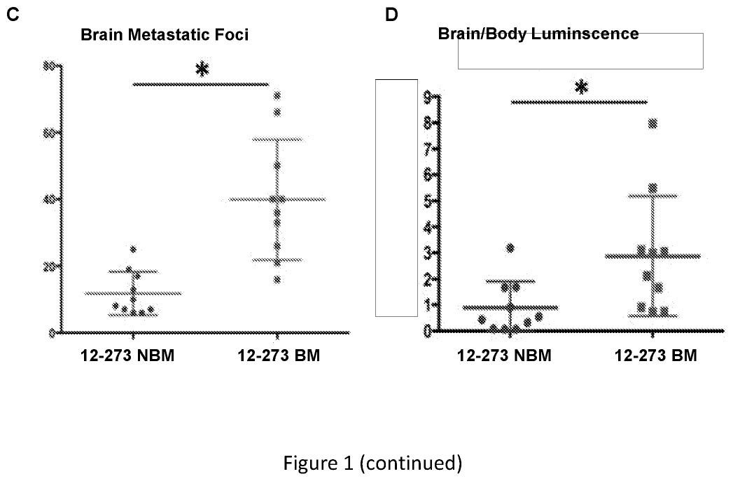

12-273 BM (brain metastasis) and NBM (non-brain metastasis) STCs were lentivirally labeled with GFP luciferase and introduced into NOG mice by intracardiac injection as illustrated in FIG. 1A. Mice were monitored weekly by IVIS imaging and were sacrificed at day 37 due to weight loss. Representative IVIS images from Day 36 (FIG. 1B) are shown. 12-273 BM vs NBM had an increased brain-to-body luminescence ratio (FIG. 1C) and an increased number of brain metastatic foci (FIG. 1D) by histological analysis, demonstrating an increased brain-specific metastatic capability.

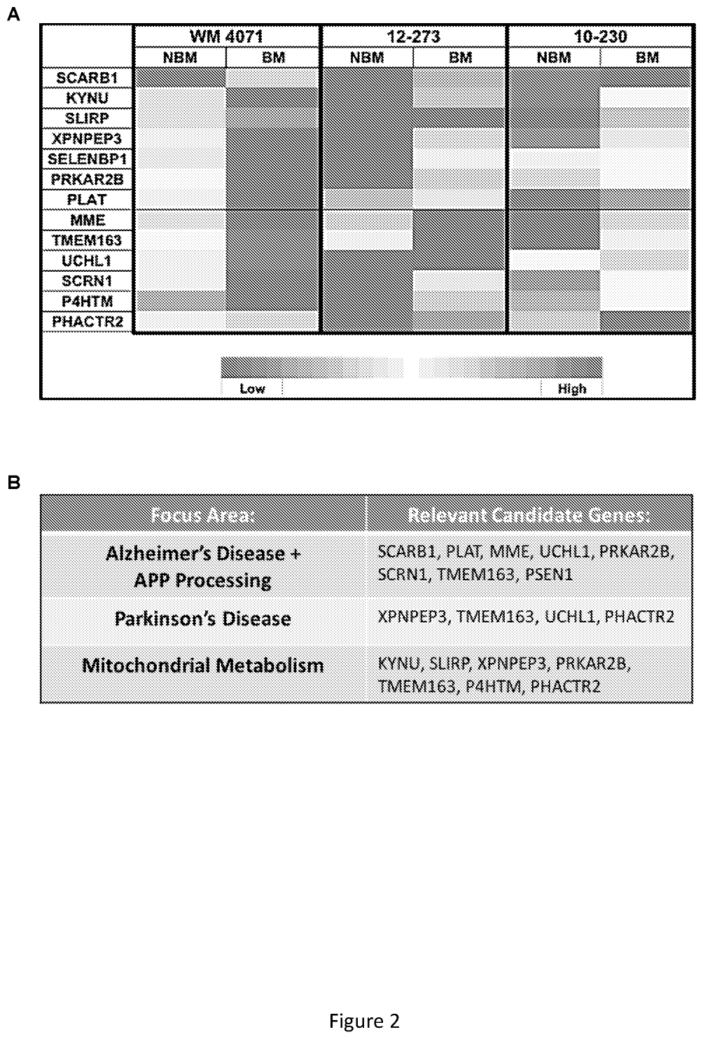

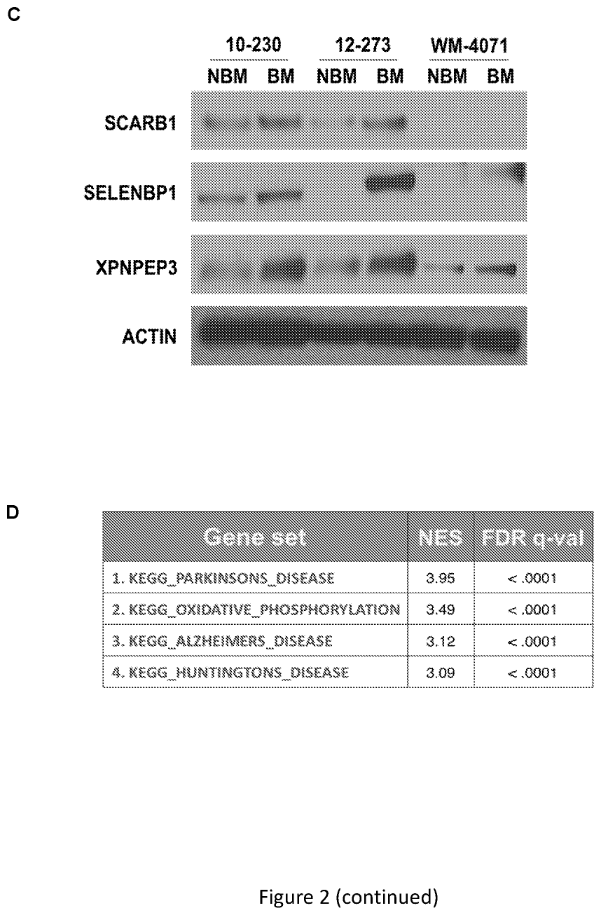

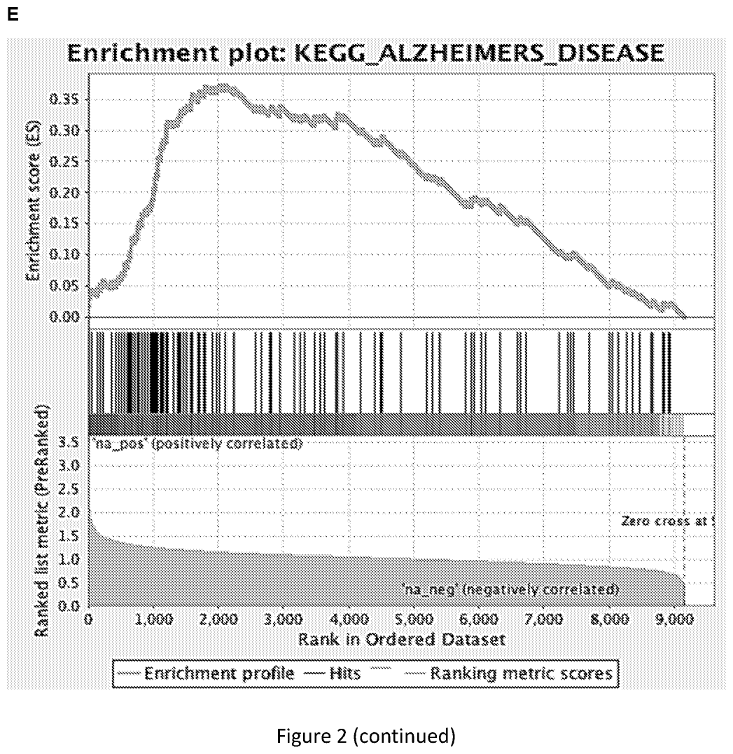

A cohort of 14 brain metastasis (BM) and 11 non-brain metastasis (NBM) derived STCs, including 3 STC pairs, were analyzed by mass spectrometry-based proteomics. Results are shown in FIG. 2. Key proteins related to neurodegeneration were found to be differentially expressed in BM vs pair NBM STCs (A, B). Select protein candidates from proteomics results were validated by western blot analysis in the STC pairs (C). Gene Set Enrichment Analysis of the proteomics from the whole cohort showed an enrichment in gene sets related to neurodegenerative pathologies (D, E). These data show consistent alterations in proteins related to neurodegenerative pathologies in BM vs NBM STCs

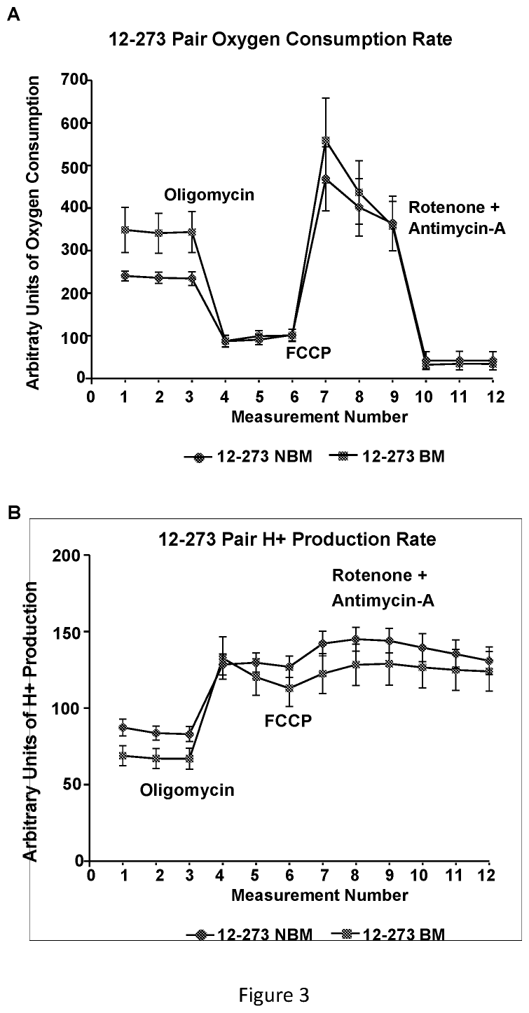

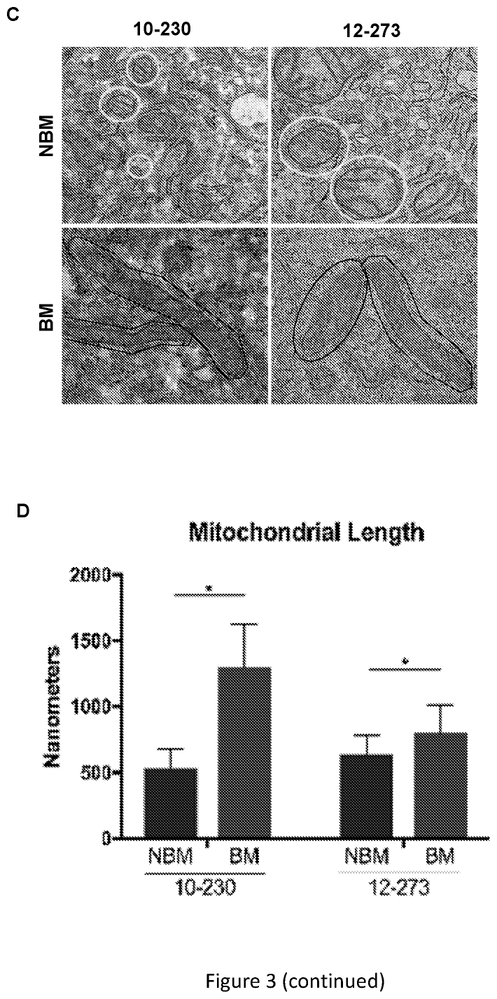

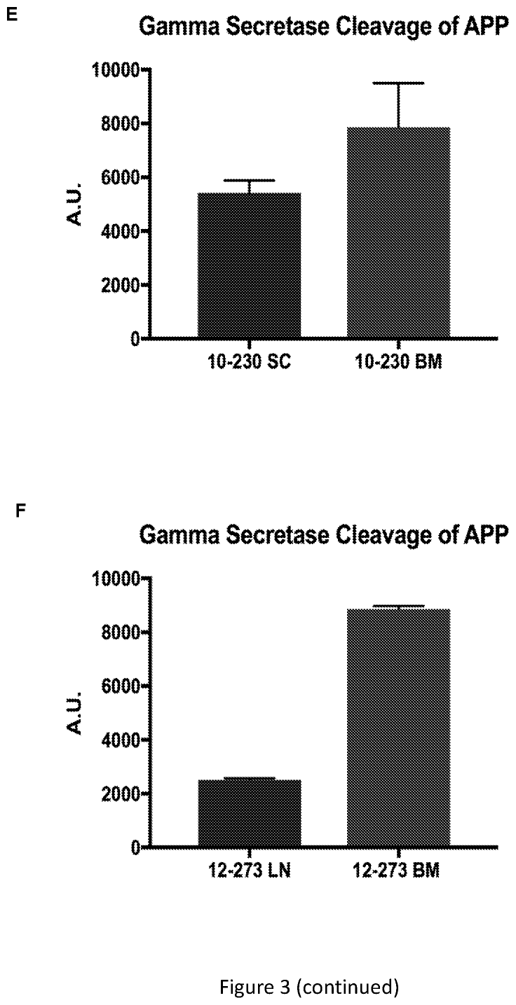

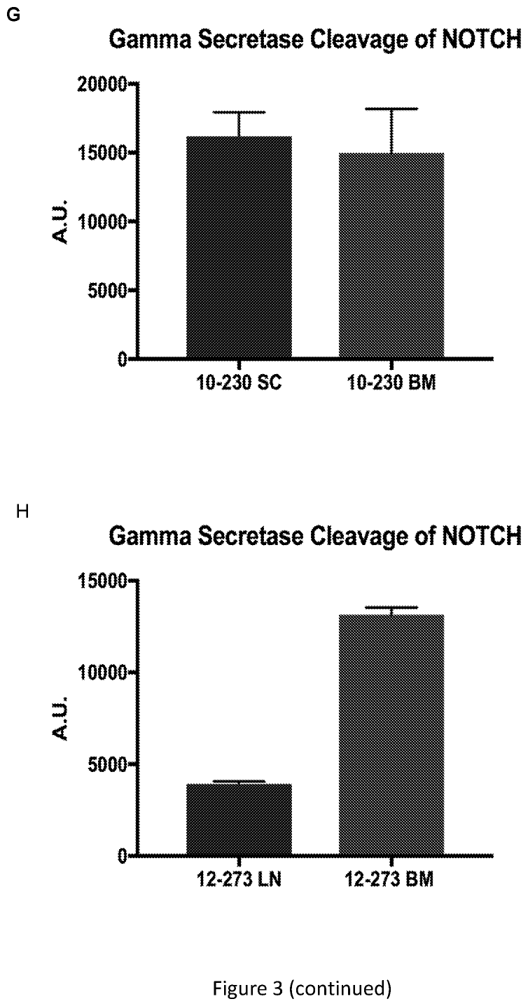

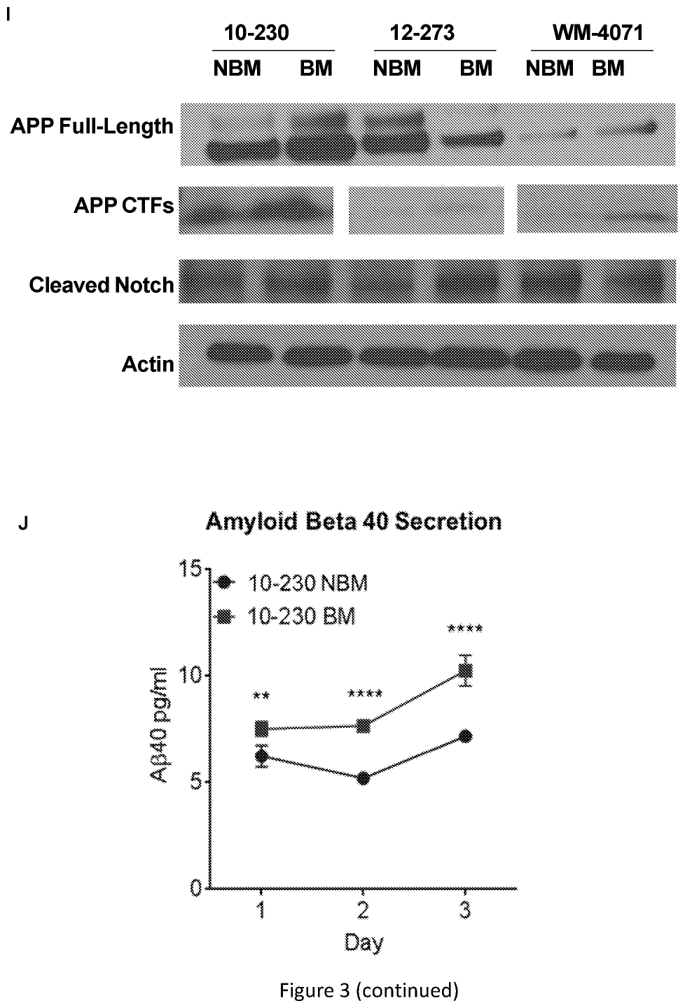

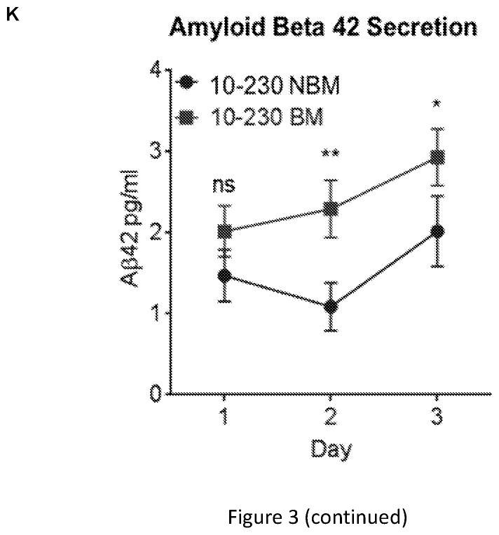

We next tested if BM compared to paired NBM have increased mitochondrial metabolism and APP processing. Results are show in FIG. 3. Analysis of STC pairs with Seahorse Mitostress test demonstrated increased basal oxidative phosphorylation (A) and decreased glycolysis (B) in BM vs pair NBM STCs (data from one pair shown). Quantification of mitochondrial length by electron microscopy was performed and showed increased mitochondrial fusion in BM vs pair NBM STCs (Representative Images C, Quantification D). Increased electron density of mitochondria in BM vs pair NBM STCs was also observed (not quantified). Substrate-specific in-vitro gamma-secretase assays were performed which demonstrated increased gamma-secretase cleavage of APP in BM vs NBM pair STCs (E, F). Interestingly, no consistent change in gamma-secretase cleavage of NOTCH was observed (G, H). Western blot analysis of whole cell lysate from STCs was performed with a C-Terminal APP Antibody which can recognize both full-length APP and the C-terminal fragments (CTFs) that remain in the membrane after APP is cleaved by alpha, beta, and/or gamma-secretase. Consistent with the results from the activity assay, APP-CTFs were consistently increased in BM vs pair NBM STCs while no consistent change was observed in cleaved NOTCH (I). No consistent change was observed in full-length APP (I). Secretion of Amyloid Beta (ABeta), which is produced by beta and gamma secretase cleavage of APP, was measured by ELISA. Secretion of both ABeta-40 (J) and Abeta-42 (K), the two predominant forms of ABeta, is increased in BM vs pair NBM STCs (data from one pair shown).

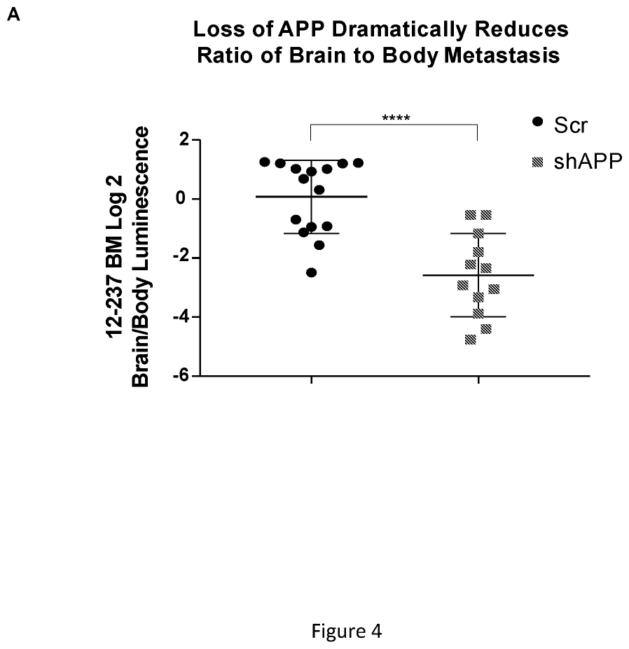

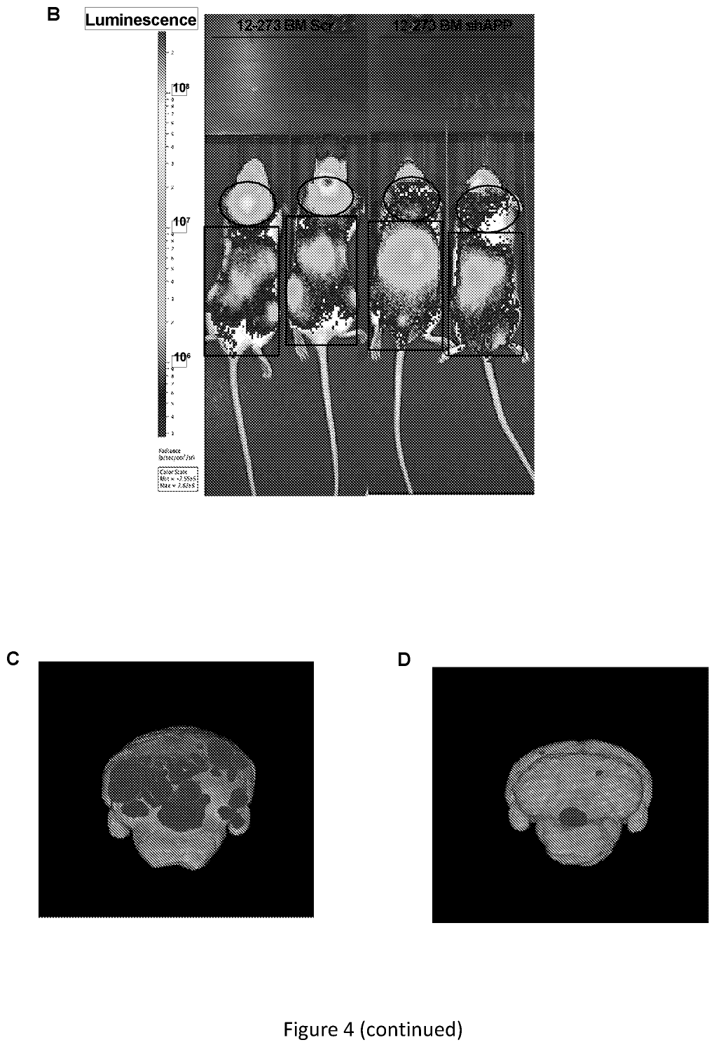

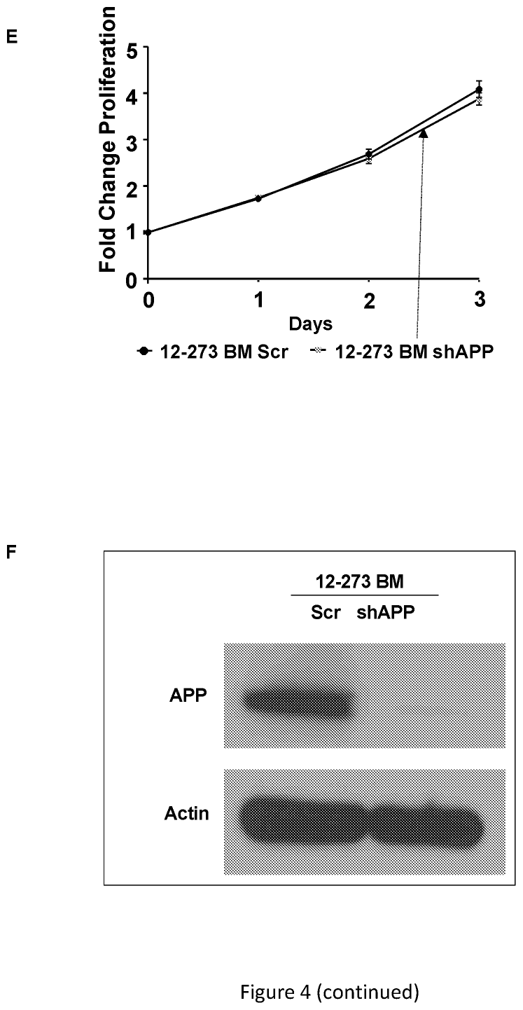

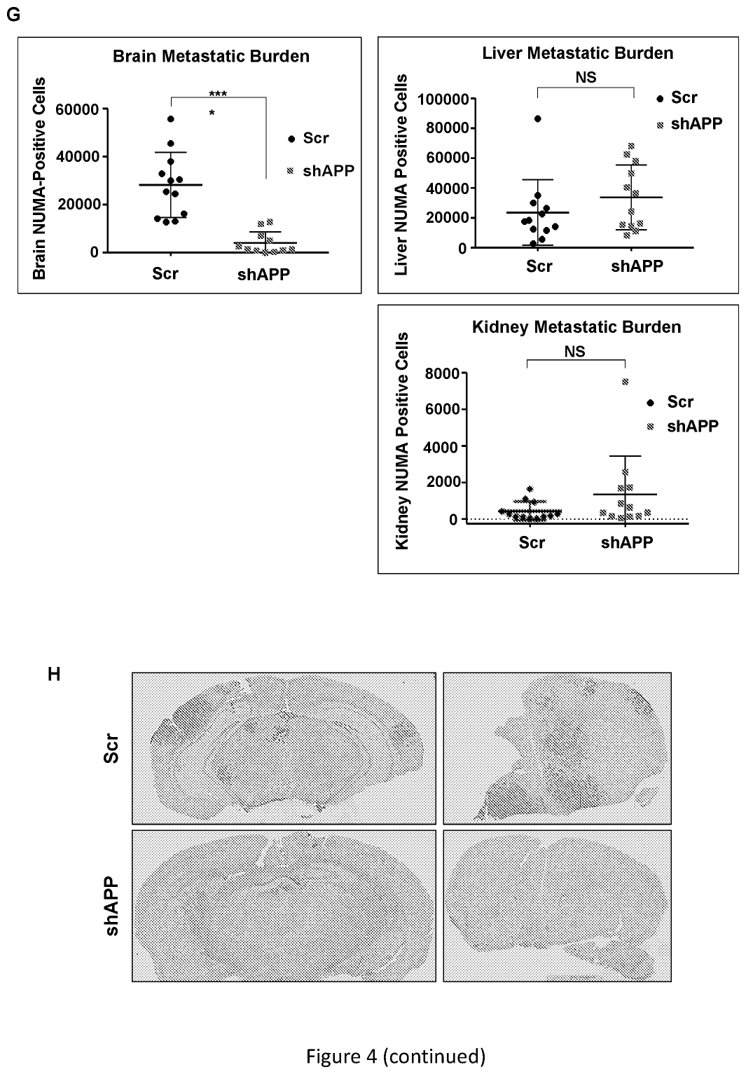

Experiments were carried out to determine if APP is required for melanoma to metastasize to the brain and to other organs. Results are shown in FIG. 4. APP was silenced by lentiviral shRNA in 12-273 BM STC and efficiency was verified by western blot analysis as compared to 12-273 BM lentiviral shRNA scramble (Scr) control (F). Silencing of APP had no significant effect on in-vitro proliferation (E). When introduced into NOG mice by intracardiac injection, silencing of APP dramatically inhibited the formation of brain metastasis without affecting metastasis to other organs (representative IVIS images B) as evidenced by a significantly decreased brain to body luminescence ratio (A). Skulls were analyzed by Mill post-mortem for detection of brain metastasis (representative images C,D). Organs were harvested and formalin-fixed, paraffin-embedded, and sectioned. Sections were stained by immunohistochemistry with an anti-NuMA antibody, which specifically recognizes human cancer cells within the mouse organs, and NuMA-positive cells were quantified using Visiopharm software. Results showed silencing of APP resulted in a severely reduced brain metastatic burden (representative images H) and no significant difference in metastatic burden to the kidney or liver (G), demonstrating that APP has a specific, critical role in melanoma brain metastasis.

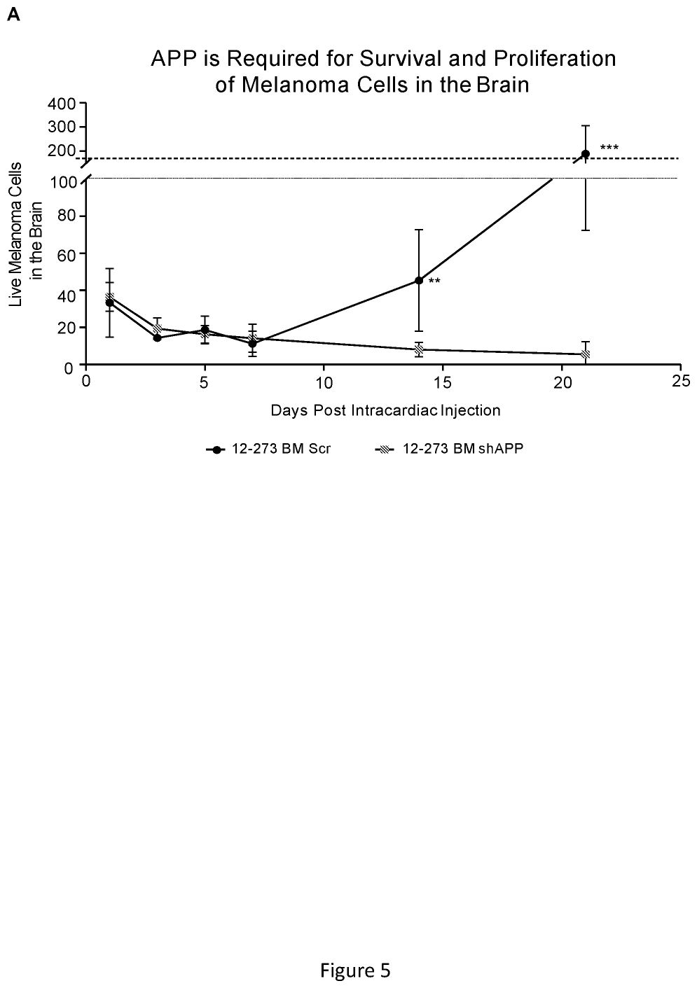

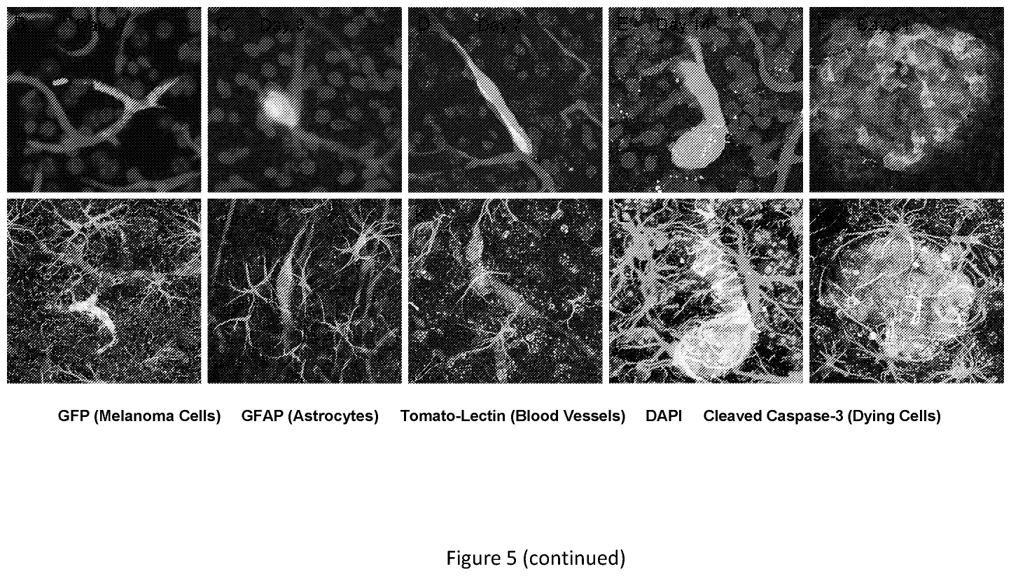

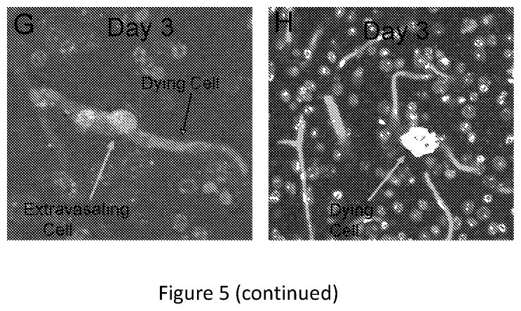

Next, we determined if APP is required for growth and survival within the brain parenchyma. 12-273 BM Scr and 12-273 BM shAPP were introduced into NOG mice by intracardiac injection. At various time points post-injection, mice were sacrificed and perfused with 4% PFA. Prior to sacrifice, mice underwent intracardiac injection with fluorescently labeled tomato-lectin, which specifically labels blood vasculature. Using a vibratome, mouse brains were sliced into 50 uM sections at resulting slices were stained for GFP, GFAP, and Cleaved Caspase-3 by free-floating immunofluorescence and slices were imaged by confocal microscopy. Results are shown in FIG. 5. At day 1, melanoma cells are confined within the blood vasculature (B). From days 2-4, melanoma cells have a rounded morphology and extravasate across the blood brain barrier (BBB) into the brain parenchyma (C). About half of cells either do not extravasate across the BBB and die (G) or die shortly after extravasation (H), as evidenced the presence of cleaved caspase-3 staining. After extravasation, surviving cells spread out along the vasculature, a phenomenon known as vascular mimicry, and begin to divide (D). By day 14, cells have started to lose contact with the vasculature and divide in a rounded morphology, leading to the formation of micrometastases (E). By day 21, small macrometastes have formed as cells continue to divide and exponentially increase in number (F). As melanoma cells begin to form brain metastases, astrocytes become reactive and have increasing levels of direct contact with the melanoma cells leading to the formation of a “glial scar” circumscribing the metastasis (I-M, GFAP Staining Days 1-21). Quantification of melanoma cells over time demonstrated that silencing of APP inhibits melanoma cell growth and survival after day 7, but does not affect BBB invasion or early survival prior to day 7 (A). All images shown are from mice injected with 12-273 BM Scr.

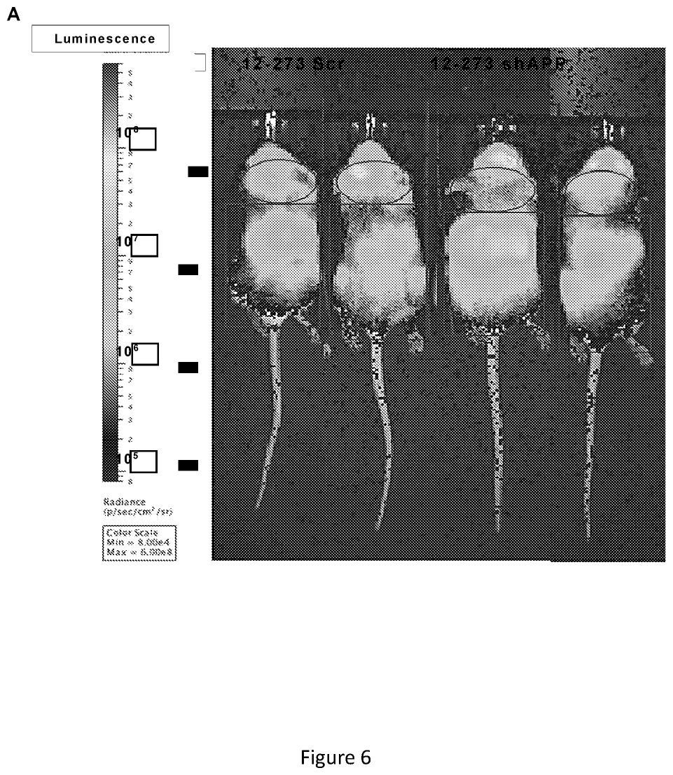

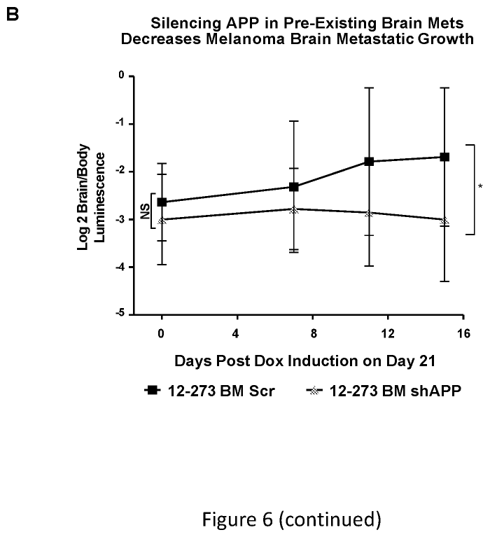

To evaluate if APP is required for the growth and survival of pre-existing brain metastases, we determined the effect of silencing APP as follows. 12-273 BM Scr and 12-273 BM shAPP were cultured in absence of doxycycline and had similar levels of APP as measured by Western Blot (data not shown) and were introduced into NOG mice by intracardiac injection. At day 21, when small macrometastases have already formed as shown in FIG. 5, silencing of APP was induced by feeding the mice doxycycline-containing food. Results are shown in FIG. 6. Silencing of APP in pre-formed brain metastases led to a significantly decreased brain metastatic burden at Day 36 (A—Representative IVIS images, B—Quantified Brain/Body luminescence), demonstrating that APP is a promising therapeutic target for the treatment of melanoma brain metastasis.

Materials and Methods

Cell Culture:

Melanoma Short Term Cultures (STCs) were prepared (de Miera et al., Pigment Cell Melanoma Research 2012, 25(3), 395-397). Melanoma STCs were cultured in DMEM supplemented with 1% Penicillin/Streptomycin, 10% Fetal Bovine Serum, and 1% MEM Non-Essential Amino Acids. All experiments performed with Melanoma STCs were carried out at passage number 40 or lower.

Proteomics Studies:

Protein from 25 Melanoma Short Term Cultures was harvested by RIPA-buffer lysis on ice for 15 minutes with vortexing every five minutes. Protein was trypsin digested and labeled using Tandem-Mass-Tag labeling which allows for multiplexing samples in a single spectrometry run. Protein samples were analyzed by mass spectrometry. Results were normalized by median peptide value. For paired STCs (a brain metastasis and a non-brain metastasis derived STC from the same patient), a log 2 BM/NBM ratio was calculated for each protein detected. For the whole STC cohort, a median BM/median NBM ratio was calculated. Proteins were sorted from highest to lowest BM/NBM ratio and Gene Set Enrichment Analysis was performed.

Lentivirus: Lentivirus was produced by transfecting 293-T cells with 3rd generation lentiviral packaging plasmids and a transfer plasmid. Virus was collected in supernatant from 293-T cells 72 hours post-transfection. Melanoma STCs cultures were then infected with lentivirus by overnight incubation in 1:2 diluted supernatant with 4 ug/mL polybrene.

Mouse studies: Melanoma STCs were lentivirally labeled with GFP luciferase. 100,000 cells were introduced into NOD. Cg-Prkdc^{scid} Il2g^{tmlWjl}/SzJ (Jackson Laboratory) by ultrasound guided injection into the left ventricle. Mice were fed either a standard diet or a diet containing 200 mg/kg doxycycline. Mice were then weekly analyzed for metastatic progression by IP injection of luciferin and IVIS luminescence imaging. At specified times or due to sufficient weight loss as defined by IUCUC guidelines, mice were injected with ketamine/xylazine and perfused with PBS and 4% paraformaldehyde.

Cellular Activities:

Oxygen Consumption Rate and Extracellular Acidification Rate in Melanoma STCs was analyzed at baseline and after injections of 1 μM oligomycin, 2 uM FCCP, and 0.5 uM antimycin-A and rotenone (according to Seahorse MitoStress Test protocol). Results were normalized to protein concentration.

Electron Microscopy:

Melanoma STCs were processed according to standard protocols for electron microscopy imaging. 20 images from random fields were taken per sample. Average mitochondrial length per sample was calculated by measuring mitochondrial length in obtained images using ImageJ software.

Gamma-Secretase Assays:

Substrate specific gamma secretase assays using APP and NOTCH substrates were performed as described in Shelton et al 2009 Molecular Neurodegeneration (ncbi.nlm.nih.gov/pubmed/19490610).

Amyloid Beta ELISA:

Levels of Amyloid beta 40 and 42 secreted from melanoma STCs were quantified using AB40 ELISA kit (Invitrogen KHB3481) and AB42 ELISA kit (Invitrogen KHB3441).

Western Blot Analysis:

Protein was harvested from melanoma STCs using RIPA buffer lysis. Levels of protein were analyzed using a standard western blot protocol. The following antibodies were used: Anti-SELENBP1 (Abcam ab90135), Anti-XPNPEP3 (Atlas HPA000527), Anti-SCARB1 (Abcam ab52629), anti-APP 22C11 (Thermofisher Scientific 14-9749-82), APP C-term antibody (751-770) (emd Millipore 171610), anti-Actin-HRP (Sigma-Aldrich A3854).

Histology and Immunohistochemistry:

Organs were formalin-fixed and paraffin embedded. 5 uM thick sections were taken from evenly spaced levels throughout organs and stained with anti-NUMA antibody, which specifically stains human melanoma cells in mouse organs. Stained sections were scanned and numbers of NUMA-positive cells were quantified using Viziopharm software analysis.

Brain Slice Immunofluorescence:

Mice were injected with fluorescently labeled tomato lectin (vector laboratories DL-1178) 4 min prior to perfusion with 4% PFA. Mouse brains were fixed overnight in PFA and then sectioned into 50 uM thick slices at four evenly spaced levels throughout the brain. Brain slices were stained by free-floating immunofluorescence with the following: Anti-GFP Alexa-488 (Santa Cruz biotechnology sc9996), Anti-GFAP (Ayes labs GFAP), Anti-Cleaved Caspase 3 Alexa-555 (cell signaling 9604S), DAPI. Live melanoma cells were quantified and images were generated using confocal microscopy.

### Example 2

This example describes an osmotic pump experiment. Alzet Osmotic Pumps (model 2006) were loaded with 1.4 μg/uL IgG MOPC21 control or an anti-Amyloid Beta antibody designated herein as Antibody 1. This is a humanized antibody which contains the same CDRs as m266 (for m266 details, see DeMattos et al., PNAS, Jul. 17, 2001, vol. 98, no. 18, pp 8850-8858, incorporated herein by reference). One skilled in the art will appreciate that humanized antibodies recognizing the same recognition sequence as m266 would bind to human amyloid beta. Pumps were attached by catheter tubing to a cannula using the Alzet Brain Infusion Kit. Pumps were then primed for 72 hours prior to implantation. Pumps were then surgically implanted for continuous infusion of antibody into the lateral left ventricle. NOD/SCID/IL2R-mice were anesthetized with a ketamine/xylazine cocktail. Using a hemostat, a subcutaneous pocket between the shoulder blades was created. The pump was placed beneath the skin in the pocket. A hole was drilled in the skull at following coordinates from bregma: 1 mm lateral, 0.3 mm posterior. The cannula was inserted at a depth of 2.5 mm and cemented into place using dental acrylic cement. The wound was subsequently closed with stiches and treated with neomycin antibiotic ointment. Mice were injected with buprenorphine for two days post-surgery. 5 to 7 days post pump implantation, 150,000 12-273 BM GFP-luciferase melanoma cells were introduced into the mice by ultrasound-guided intracardiac injection in the left ventricle. Metastatic progression in the brain and the body were monitored weekly by IVIS imaging after injection of mice intraperitoneally with luciferin.

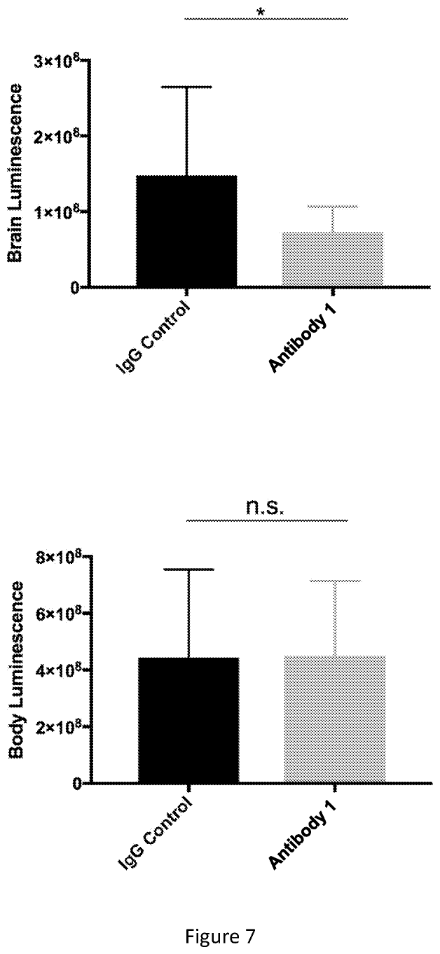

As shown in FIG. 7, mice treated with Antibody 1 have significantly decreased brain tumor signal (around 50% of control) at day 25 while exhibiting no difference in body tumor signal when compared to control IgG treated mice.

### Example 3

This example demonstrates that amyloid beta is the required form of APP for melanoma brain metastasis.

Methods:

Gamma secretase activity assay (a and b) was carried out as described above.

Amyloid Beta ELISA (c,f): Amyloid Beta-40 ELISA was performed using a kit from Thermo Fisher Scientific (Catalogue #: KHB3481). 1 mL of media was exposed to 90% confluent melanoma STCs is 6 well plates for 24 hours. Conditioned media was removed and then concentrated 10× using Amicon Ultra 3 kDA 0.5 ml Centrifugal Filter Unit (Millipore Sigma Catalogue: UFC500396). 50 ul of concentrated CM was added per well of sample in duplicates.

Cloning (d): A plasmid containing APP-770 cDNA (accession number NM 000484.3) was purchased from genocopoeia and was transferred into pLVX-iRES td-Tomato lentiviral vector using restriction enzyme cloning. SPA4CT-T43P was cloned by creating custom oligos that were annealed to form the SPA4CT-T43P insert with restriction digest overhangs, which was subsequently ligated into pLVX-iRES td-Tomato lentiviral vector. Lentivirus generation and cell infection was carried out as described above. Western blot analysis was carried out as described above.

Antibodies (d,e): anti-APP 22C11 was purchased commercially from Thermo Fisher Scientific (Catalogue #: 14-9748072). APP C1/6.1 was synthesized and provided as a gift from the Paul Mathews laboratory at NYU.

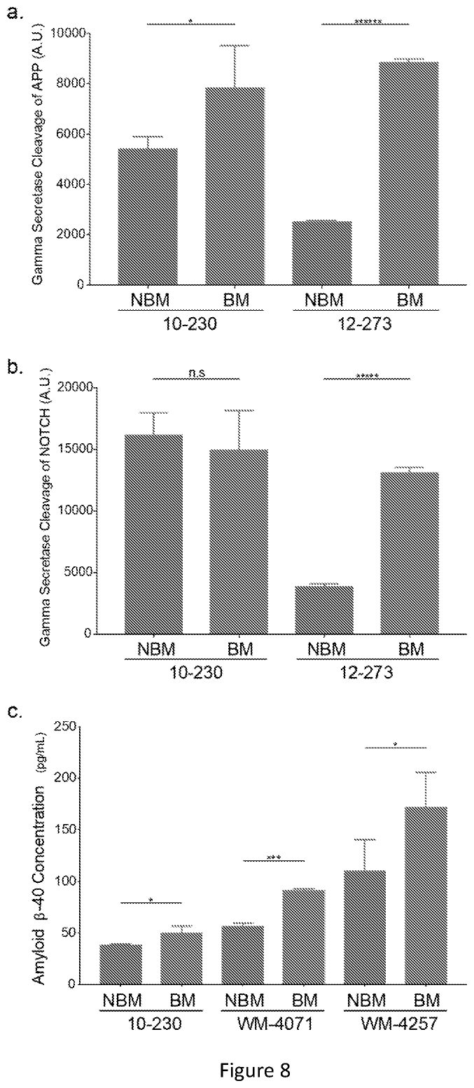

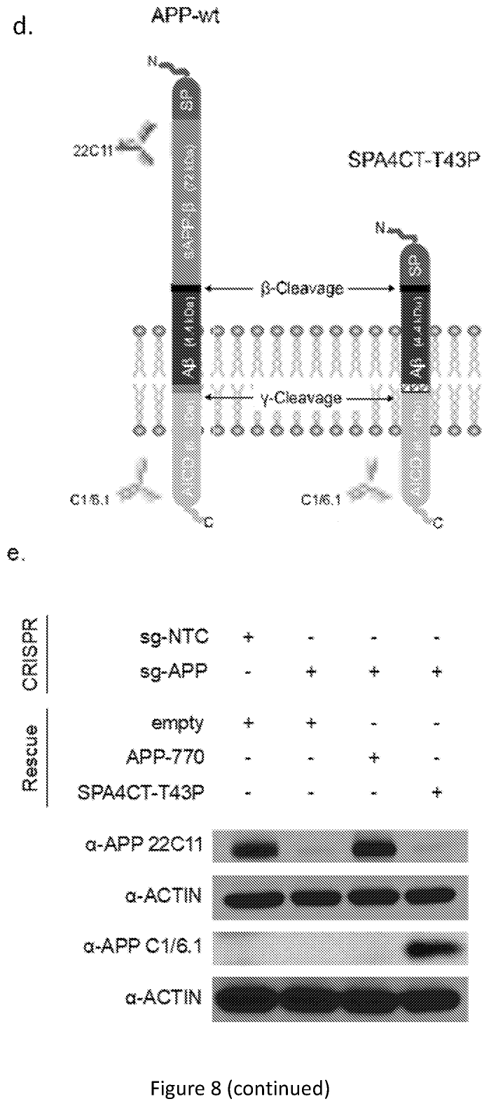

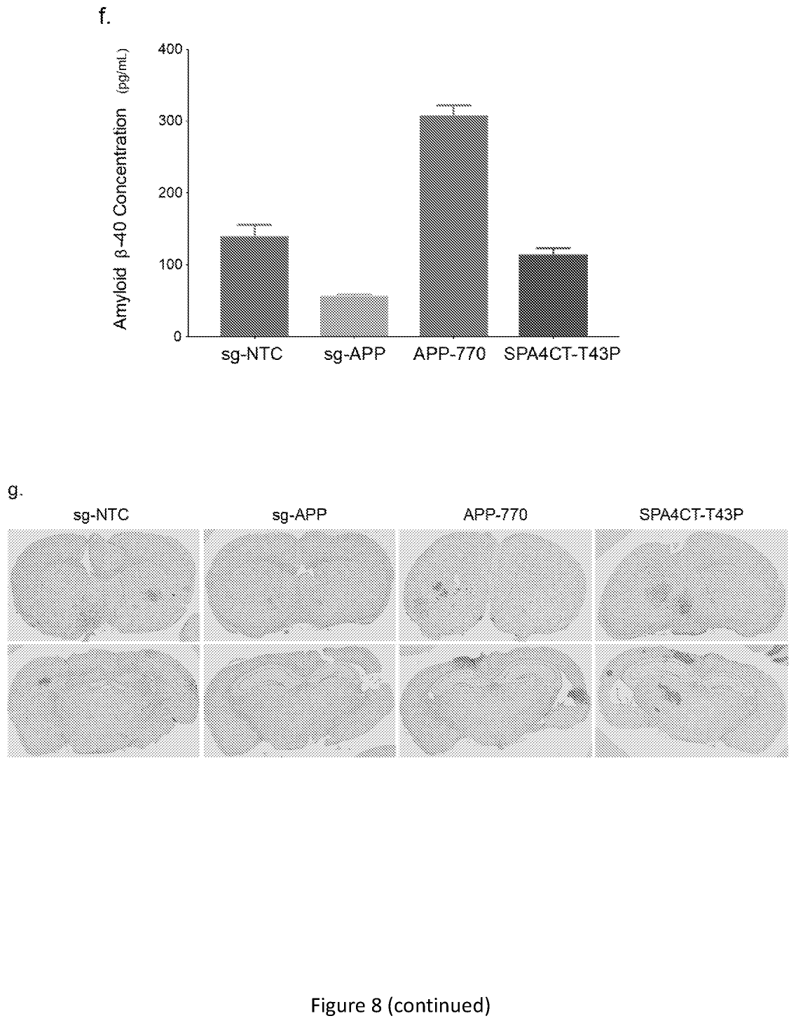

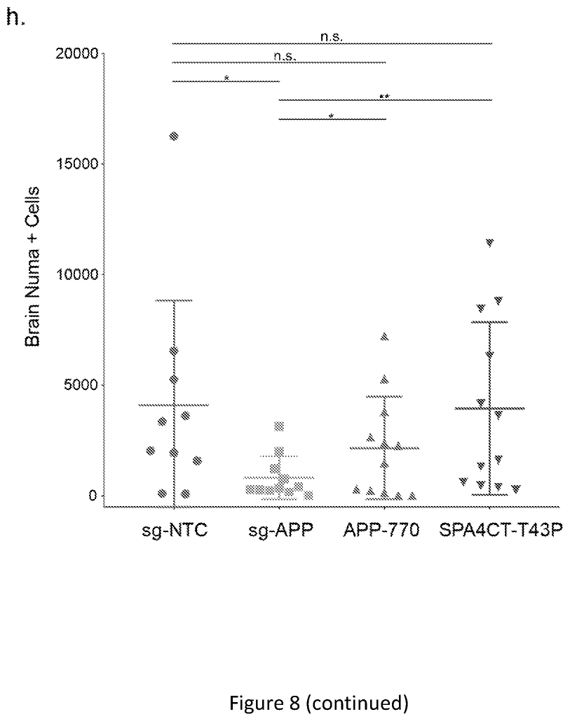

Intracardiac injection and quantification of met burden by NUMA staining (g and h) was carried out as described above. The results are described in the Figure legend for FIG. 8. Probe-specific gamma secretase assays showed that pair BM vs NBM STCs had consistently increased gamma secretase cleavage of APP (a) but not of NOTCH (b). Amyloid Beta-40 ELISAs demonstrated the pair BM vs NBM STCs have increased secretion of amyloid beta (c). APP knock out using CRISPR-Cas9 and wild-type APP and SPA4CT-T43P was re-expressed and expression was verified by western blot (e). SPA4CT-T43P expression APP knockout cells led to amyloid beta secretion at comparable levels to NTC control cells (f). Upon intracardiac injection, APP knockout cells (sg-APP) had a reduced ability to metastasize to the brain when compared with NTC control cells (g-h). Full length, wild-type APP re-expression in APP knockout cells was able to resuce the defects observed in brain metastasis (g-h). SPA4CT-T43P re-expression was also able to rescue the defects observed in brain metastasis with APP knockout (g-h), demonstrating that amyloid beta is the required form of APP for melanoma brain metastasis.

While the present invention has been described through illustrative embodiments, routine modification will be apparent to those skilled in the art and such modifications are intended to be within the scope of this disclosure.

What is claimed is:

1. A method of treating cancer brain metastases comprising administering to an individual in need of treatment a composition comprising one or more inhibitors of amyloid precursor protein (APP) processing in the brain.

2. The method of claim 1, wherein the APP processing is enzymatic cleavage.

3. The method of claim 2, wherein the inhibitor of APP enzymatic cleavage is an inhibitor of α-, β-, or γ-secretase.

4. The method of claim 3, wherein the inhibitor is γ-secretase and the γ-secretase does not have a significant effect on Notch activity.

5. A method of treating or preventing cancer brain metastases comprising administering to an individual in need of treatment a composition comprising an inhibitor of an APP enzymatic cleavage product in the brain.

6. The method of claim 5, wherein the APP enzymatic cleavage product is secreted APP α (sAPPα), secreted APP β (sAPPβ), carboxyterminal fragment 83 (CTF83), carboxyterminal fragment 99 (CTF99), p3, amyloid β-peptide (Aβ), or amino-terminal APP intracellular domain (AICD).

7. The method of claim 6, wherein the inhibitor is an antibody specific for one or more of sAPPα, sAPPβ, CTF83, CTF99, p3, Aβ, and AICD.

8. The method of claim 6, wherein the APP cleavage product is Aβ, and the inhibitor is an anti-amyloid beta (Aβ) antibody.

9. The method of claim 8, wherein the anti-Aβ antibody specifically binds to the monomeric form of Aβ.

10. The method of claim 4, wherein the anti-Aβ antibody preferentially binds to the monomeric form of Aβ over the oligomeric form of Aβ.

11. The method of claim 1, wherein the cancer is melanoma.

12. The method of claim 1, wherein the cancer is lung cancer or breast cancer.


SEQUENCE LISTING (8 sequences)

SEQ ID NO: 1 - protein, 40 aa, Homo sapiens
DAEFRHDSGYEVHHQKLVFFAEDVGSNKGAIIGLMVGGVV

SEQ ID NO: 2 - protein, 42 aa, Homo sapiens [sequence omitted]

SEQ ID NO: 3 - protein, 670 aa, Homo sapiens
LEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQK

SEQ ID NO: 4 - protein, 654 aa, Homo sapiens
LEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKM

SEQ ID NO: 5 - protein, 99 aa, Homo sapiens
DAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 6 - protein, 83 aa, Homo sapiens
LVFFAEDVGSNKGAIIGLMVGGVVIATVIVITLVMLKKKQYTSIHHGVVEVDAAVTPEERHLSKMQQNGYENPTYKFFEQMQN

SEQ ID NO: 7 - protein, 26 aa, Homo sapiens
LVFFAEDVGSNKGAIIGLMVGGVVIA

SEQ ID NO: 8 - protein, 59 aa, Homo sapiens
IATVIVITLVMLKKKQYTSIHHGVVEVDAAVTPEERHLSKMQQNGYENPTYKFFEQMQN